(19) United States
(12) Patent Application Publication
(10) Pub. No.: US 2026/0334805 A1
(43) Pub. Date: Oct. 29, 2026

(54) APPARATUS AND METHOD FOR MEASURING ELECTRODERMAL ACTIVITY WITH AMBIENT TEMPERATURE-ADAPTIVE AUTOMATIC GAIN CONTROL AND AUTOMATIC CALIBRATION FUNCTION

(71) Applicant: ELECTRONICS AND TELECOMMUNICATIONS RESEARCH INSTITUTE, Daejeon (KR)

(72) Inventors: Seung Yoon Nam, Daejeon (KR); Chanki Park, Daejeon (KR); HyunSoon Shin, Daejeon (KR)

(21) Appl. No.: 19/374,156

(22) Filed: Oct. 30, 2025

(30) Foreign Application Priority Data
Apr. 29, 2025 (KR) 10-2025-0056271

(51) Int. Cl. A61B 5/0531; A61B 5/00

(52) U.S. Cl. CPC A61B 5/0531; A61B 5/6813; A61B 5/7225; A61B 5/7271; A61B 2560/0223; A61B 2560/0252; A61B 2562/0271

(57) ABSTRACT

Disclosed are an apparatus and method for measuring electrodermal activity with an ambient temperature-adaptive automatic gain control and automatic calibration function without an LUT. The apparatus includes a temperature measuring unit including a temperature sensor for measuring an ambient temperature, an amplification circuit unit configured to amplify a signal, a MOSFET configured to input a current to the amplification circuit unit, and a controller configured to apply a gate voltage to a gate of the MOSFET based on the ambient temperature from the temperature measuring unit and an output voltage of the amplification circuit unit. The controller measures the output voltage of the amplification circuit unit, estimates a MOSFET current that flows from a drain of the MOSFET to a source of the MOSFET based on the applied gate voltage, and calculates electrodermal activity based on the measured output voltage and the estimated MOSFET current.

### CROSS-REFERENCE TO RELATED APPLICATION(S)

This application claims priority from and the benefit of Korean patent application No. 10-2025-0056271, filed on Apr. 29, 2025, which is hereby incorporated by reference for all purposes as if set forth herein.

### BACKGROUND

1. Technical Field

The present disclosure relates to an apparatus and method for measuring electrodermal activity with an ambient temperature-adaptive automatic gain control and automatic calibration function.

2. Related Art

Electrodermal activity is one of physiological signals and has a close relation with the activity of sweat gland. The results of the analysis of a change in the electrodermal activity signal may be used as an index for the negative effects of a living body, such as stress, pain, and an arousal state. The electrodermal activity signal has different ranges, such as a height and amplitude depending on a person. The reason for this is that the state of skin differently appears depending on an environment or a sensitive situation. For such a reason, the electrodermal activity signal is measured in a wide range of 0.01 uS to 80 uS.

The electrodermal activity signal may be divided into a skin conductance level (CL), that is, a low frequency component, and a skin conductance response (SCR), that is, a high frequency component. In general, amplitude of an SCL signal is a range of 0.5 to 80 uS, and amplitude of the SCR signal is a range of 1 to 5 uS.

Representatively, a Wheatstone bridge method, a voltage source driving method, and a current source driving method are used in a circuit that measures the electrodermal activity. Among the methods, the voltage source driving method and the current source driving method are widely used because the methods are simple in a circuit configuration and are easy to calculate the electrodermal activity. DC or AC may be the driving source.

It is worth noting that the magnitude of the electrodermal activity signal varies significantly not only due to the response of an autonomic nervous system, but also depending on an individual difference in sweat secretion, temperature, and humidity. There may be up to a 1000-fold difference in the magnitude of the electrodermal activity signal. For such a reason, when a circuit gain is low, the sensitivity of the signal becomes excessively high, making it difficult to measure subtle variations. When the circuit gain is high, there is a problem in that the signal may be easily saturated. Although such a problem may be alleviated to some extent by reducing the circuit gain and employing a high-performance analog-to-digital converter (ADC), the use of a high-performance ADC is costly and furthermore unsuitable for wearable applications.

In a conventional MOSFET-based apparatus for measuring electrodermal activity, such as that illustrated in FIG. 6, a MCU 11 controls a current by adjusting the gate voltage of an N-MOSFET 12. An output voltage vₒᵤₜ amplified by an amplifier 13 is measured. Electrodermal activity is calculated from the measured value. To this end, N-MOSFET characteristics relating to a relation of the gate voltage and the current are stored in a look-up table (LUT), and a circuit is controlled by using the stored N-MOSFET characteristics.

In order to measure the electrodermal activity with a high dynamic range and high resolution, an AGC function capable of precise control is required. A conventional MOSFET-based AGC is controlled by the output of a DAC. However, the output of the DAC ranges from 0 to Vcc, whereas the range of a voltage rage that is necessary for the gate of the MOSFET 130 is relatively narrow (e.g., 2 to 3 V). Accordingly, resolution that actually controls the gate voltage of the MOSFET is reduced. For example, if only 2 to 3 V of 0 to 5 V is used, DAC resolution that is used to control the circuit is reduced from 1000 to 200, for example.

The characteristics of the MOSFET may vary depending on factors such as manufacturing tolerances and temperature changes. In such a case, if a value that is initially stored in the LUT is used, there is a problem in that significant errors may occur because variations caused by manufacturing tolerances and ambient temperature are not compensated.

### SUMMARY

Various embodiments are directed to measuring a change in a MOSFET current attributable to an ambient temperature change in real time and measuring an accurate electrodermal activity signal without an LUT.

Various embodiments are directed to improving the accuracy and utilization of an electrodermal activity signal by optimizing signal sensitivity and a measuring range through an automatic gain control function of an amplifier.

An apparatus for measuring electrodermal activity according to an embodiment of the present disclosure includes a temperature measuring unit including a temperature sensor for measuring an ambient temperature, an amplification circuit unit configured to amplify a signal, a MOSFET configured to input a current to the amplification circuit unit, and a controller configured to apply a gate voltage to a gate of the MOSFET based on the ambient temperature from the temperature measuring unit and an output voltage of the amplification circuit unit. The controller measures the output voltage of the amplification circuit unit, estimates a MOSFET current that flows from a drain of the MOSFET to a source of the MOSFET based on the applied gate voltage, and calculates electrodermal activity based on the measured output voltage and the estimated MOSFET current.

In an embodiment, the amplification circuit unit includes an amplifier, a calibration resistor connected in parallel to the amplifier and for current modeling, skin electrodes attached to skin and configured to measure electrodermal activity, and a switch configured to select one of the calibration resistor and the skin electrodes. The controller connects the switch to the calibration resistor, generates a current model, and measures the electrodermal activity by using the current model by connecting the switch to the skin electrodes.

In an embodiment, the controller generates the current model by performing connecting the switch to the calibration resistor, measuring the ambient temperature through the temperature measuring unit, applying the gate voltage to a gate of the MOSFET while changing the gate voltage and measuring the output voltage, calculating the MOSFET current based on the measured output voltage and resistance of the calibration resistor, and modeling a nonlinear relation of the MOSFET current to the gate voltage of the MOSFET, based on the measured ambient temperature, the applied gate voltage, the output voltage measured with respect to the applied gate voltage, and the calculated MOSFET current.

In an embodiment, the controller applies the gate voltage to the gate of the MOSFET while raising the gate voltage by a predetermined unit voltage with respect to the entire operating range of the MOSFET and measures the output voltage at that time.

In an embodiment, the controller may use polynomial-based modeling and a parameter estimation method when modeling the nonlinear relation of the MOSFET current to the gate voltage of the MOSFET.

In an embodiment, the controller may measure the electrodermal activity by performing a step of selecting the skin electrodes by connecting the switch to the skin electrodes, a step of measuring the ambient temperature by using the temperature measuring unit, an output voltage measuring step of applying the gate voltage within an operating range of the MOSFET and measuring the output voltage, an electrodermal activity calculation step of calculating the MOSFET current based on the measured value and the current model and calculating the electrodermal activity based on the measured output voltage value and the calculated MOSFET current, a gate voltage adjustment step of adjusting the gate voltage based on the measured output voltage, and a step of measuring the electrodermal activity while repeating the output voltage measuring step, the electrodermal activity calculation step, and the gate voltage adjustment step.

In an embodiment, the controller checks the calculated electrodermal activity and adjusts the gate voltage so that a gain is adjusted to a predetermined range of a current signal while increasing the sensitivity of the electrodermal activity, in the gate voltage adjustment step. The predetermined range of the current signal may be a range of 50% to 150% of the current signal.

A method of measuring electrodermal activity according to an embodiment of the present disclosure is a method of measuring electrodermal activity in an apparatus for measuring electrodermal activity, including a temperature measuring unit including a temperature sensor for measuring an ambient temperature, an amplification circuit unit configured to amplify a signal, a MOSFET configured to input a current to the amplification circuit unit, and a controller configured to apply a gate voltage to a gate of the MOSFET based on the ambient temperature from the temperature measuring unit and an output voltage of the amplification circuit unit. The amplification circuit unit includes an amplifier, a calibration resistor for current modeling, skin electrodes attached to skin and configured to measure electrodermal activity, and a switch configured to select one of the calibration resistor and the skin electrodes. The method includes a current model generation step of connecting, by the controller, the switch to the calibration resistor and generating a current model, and an electrodermal activity measuring step of connecting, by the controller, the switch to the skin electrodes and measuring electrodermal activity by using the current model.

In the electrodermal activity measuring step according to an embodiment of the present disclosure, the controller may connect the switch to the skin electrodes, applies the gate voltage to a gate of the MOSFET, may measure the output voltage of the amplification circuit unit, estimates MOSFET current that flows from a drain of the MOSFET to a source of the MOSFET by using the current model based on the applied gate voltage, and may calculate the electrodermal activity based on the measured output voltage and the estimated MOSFET current.

In an embodiment, the current model generation step includes a step of connecting the switch to the calibration resistor, a step of measuring the ambient temperature through the temperature measuring unit, an output voltage measuring step of applying the gate voltage to the gate of the MOSFET while changing the gate voltage and measuring the output voltage, a step of calculating the MOSFET current based on the measured output voltage and resistance of the calibration resistor, and a modeling step of modeling a nonlinear relation of the MOSFET current to the gate voltage of the MOSFET based on the measured ambient temperature, the applied gate voltage, the output voltage measured with respect to the applied gate voltage, and the calculated MOSFET current.

In an embodiment, in the output voltage measuring step, the controller applies the gate voltage to the gate of the MOSFET while raising the gate voltage by a predetermined unit voltage with respect to the entire operating range of the MOSFET and measures an output voltage at that time.

In an embodiment, in the modeling step, the controller models a nonlinear relation of the MOSFET current to the gate voltage of the MOSFET by using polynomial-based modeling and a parameter estimation method.

In an embodiment, the electrodermal activity measuring step includes a step of selecting the skin electrodes by connecting the switch to the skin electrodes, a step of measuring the ambient temperature by using the temperature measuring unit, an output voltage measuring step of applying the gate voltage within an operating range of the MOSFET and measuring the output voltage, an electrodermal activity calculation step of calculating the MOSFET current by using the measured value and the current model and calculating the electrodermal activity based on the measured output voltage and the calculated MOSFET current, a gate voltage adjustment step of adjusting the gate voltage based on the measured output voltage, and a step of measuring the electrodermal activity while repeating the output voltage measuring step, the electrodermal activity calculation step, and the gate voltage adjustment step.

In the gate voltage adjustment step, the controller may check the calculated electrodermal activity and may adjust the gate voltage so that a gate is adjusted to a predetermined range of a current signal while increasing the sensitivity of the electrodermal activity. In an embodiment, the predetermined range of the current signal is a range of 50% to 150% of the current signal.

According to an embodiment of the present disclosure, it is possible to improve the reliability of the measurement of an electrodermal activity signal through calibration for a temperature change. That is, the accuracy and reliability of the electrodermal activity signal can be greatly improved through an accurate MOSFET current Ids by calibrating MOSFET characteristics that are changed according to a temperature in real time by measuring the temperature in real time.

Furthermore, according to an embodiment of the present disclosure, efficient memory management can be achieved. In a conventional method of measuring electrodermal activity using a MOSFET, the MOSFET current Ids for the entire operating range needs to be stored in the LUT. In contrast, in an embodiment of the present disclosure, memory can be efficiently used because only a set of derived parameters is stored.

Furthermore, according to an embodiment of the present disclosure, an electrodermal activity signal can be continuously measured. In an embodiment, of the present disclosure, the LUT for the MOSFET current Ids can be used without being updated periodically with respect to a temperature that is changed in real time because parameters are initially derived through one modeling. Accordingly, there is no problem in that the electrodermal activity signal is stopped because there is no process of measuring the MOSFET current Ids again.

Furthermore, according to an embodiment of the present disclosure, it is possible to maintain optimal sensitivity and a stable value through automatic gain control. In the case of electrodermal activity, in which the signal amplitude varies significantly depending on individual differences and conditions, AGC is required in order to secure ADC resolution. In an embodiment of the present disclosure, sensitivity that is changed depending on a gain and the saturation of a signal or a clipping phenomenon do not occur because the size of a signal (gain) is adjusted by predicting a fixed range of a current signal value.

Effects of the present disclosure which may be obtained in the present disclosure are not limited to the aforementioned effects, and other effects not described above may be evidently understood by a person having ordinary knowledge in the art to which the present disclosure pertains from the following description.

### BRIEF DESCRIPTION OF THE DRAWINGS

FIG. 1 is a block diagram illustrating a construction of an apparatus for measuring electrodermal activity according to an embodiment of the present disclosure.

FIG. 2 is a flowchart illustrating a process of performing MOSFET current modeling and measuring electrodermal activity by using the MOSFET current modeling in the apparatus for measuring electrodermal activity according to an embodiment of the present disclosure.

FIG. 3 is a flowchart illustrating a process of modeling a MOSFET current that flows into skin with respect to an ambient temperature and a gate voltage applied to a MOSFET according to an embodiment of the present disclosure.

FIG. 4 is a flowchart illustrating a process of measuring electrodermal activity in an embodiment of the present disclosure.

FIG. 5 is a flowchart illustrating a process of adjusting an optimal sensitivity and range based on an extracted electrodermal activity value.

FIG. 6 illustrates an example of a conventional apparatus for measuring electrodermal activity using a MOSFET.

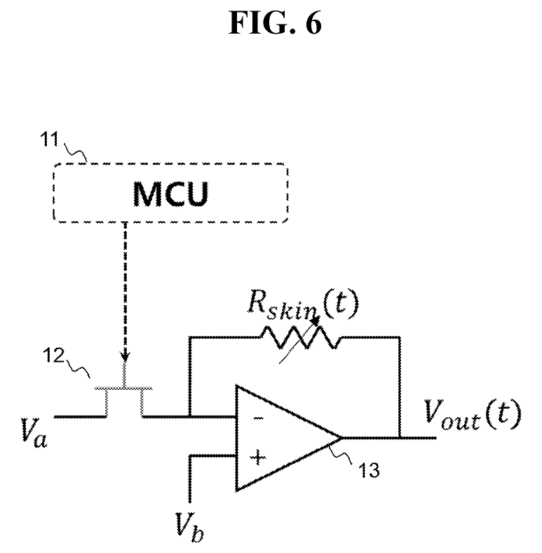

### DETAILED DESCRIPTION

The aforementioned object, other objects, advantages, and characteristics of the present disclosure and a method for achieving the objects, advantages, and characteristics will become clear with reference to embodiments to be described in detail along with the accompanying drawings.

However, the present disclosure is not limited to embodiments disclosed hereinafter, but may be implemented in various different forms. The following embodiments are merely provided to easily notify a person having ordinary knowledge in the art to which the present disclosure pertains of the objects, constructions, and effects of the present disclosure. The scope of rights of the present disclosure is defined by the writing of the claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other components, steps, operations and/or components in addition to mentioned components, steps, operations and/or components.

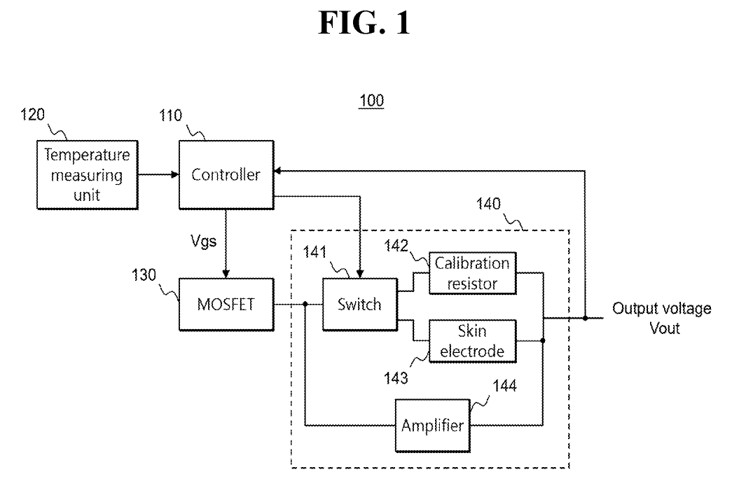

FIG. 1 is a block diagram illustrating a construction of an apparatus for measuring electrodermal activity according to an embodiment of the present disclosure. The apparatus 100 for measuring electrodermal activity in FIG. 1 includes a temperature measuring unit 120 including a temperature sensor for measuring an ambient temperature, a MOSFET 130, an amplification circuit unit 140 that amplifies a signal based on an input current or voltage from the MOSFET, and a controller 110 that outputs a gate voltage Vgs of the MOSFET based on a temperature from the temperature measuring unit 120 and the output voltage of the amplification circuit unit 140. The controller 110 calculates electrodermal activity based on a measured output voltage Vout and a MOSFET current Ids that is estimated based on an applied gate voltage Vgs and that flows from the drain of the MOSFET to the source of the MOSFET.

The amplification circuit unit 140 includes an amplifier 144, a calibration resistor 142 for current modeling, skin electrodes 143 attached to skin and configured to measure electrodermal activity, and a switch 141 for selecting the calibration resistor 142 and the skin electrodes 143. The controller 110 controls the switch 141 to be connected to the calibration resistor 142 upon current modeling and to be connected to the skin electrodes 143 upon measurement of electrodermal activity.

The MOSFET 130 that controls the gain of the amplifier and a current that flows into the skin in order to measure electrodermal activity includes the gate, the source, and the drain, and may control the MOSFET current Ids that flows from the drain to the source based on the gate voltage Vgs. However, although the same gate voltage Vgs is applied the MOSFET, the size of the MOSFET current Ids may be different depending on an ambient temperature and a temperature “t” of the MOSFET 130.

Accordingly, if information on the temperature is unknown, it is impossible to calculate an accurate MOSFET current Ids and thus impossible to calculate accurate electrodermal activity. Therefore, the electrodermal activity needs to be calculated based on the accurate MOSFET current Ids for information on the temperature. In an embodiment of the present disclosure, in order to solve such a problem, current modeling is performed by using the calibration resistor 142. The skin electrodes 143 is used to measure electrodermal activity after the current modeling. The calibration resistor 142 and the skin electrodes 143 are connected in parallel. One of the calibration resistor 142 and the skin electrodes 143 is selected by using the switch 141 that is connected to the calibration resistor 142 and the skin electrodes 143 in series. For example, a MUX/DEMUX switch may be used as the switch 141.

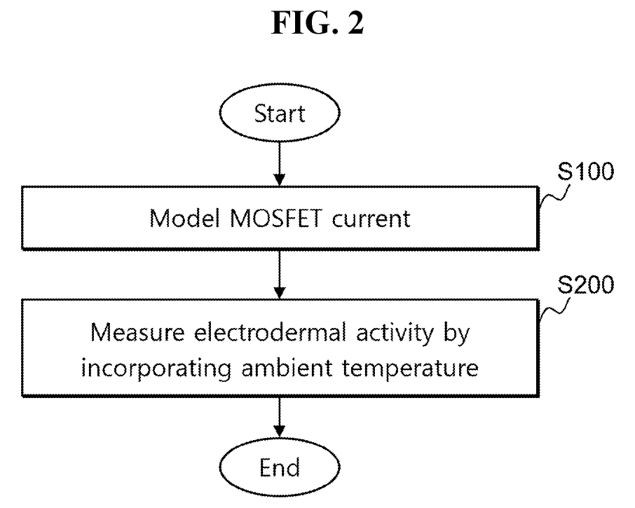

FIG. 2 is a flowchart illustrating a process of performing MOSFET current modeling and measuring electrodermal activity by using the MOSFET current modeling in the apparatus for measuring electrodermal activity according to an embodiment of the present disclosure. First, the controller 110 connects the switch 141 to the calibration resistor 142 and models a MOSFET current that flows into the skin with respect to the ambient temperature “t” and the gate voltage Vgs that is applied to the MOSFET (step S100). Step S100 may be performed when the apparatus is used for the first time or upon power-on. According to an embodiment, step S100 may be performed based on a user’s modeling request manipulation. When measuring electrodermal activity, the controller 110 connects the switch 141 to the skin electrodes 143, and measures the electrodermal activity by incorporating an ambient temperature based on a current model generated in step S100 (step S200).

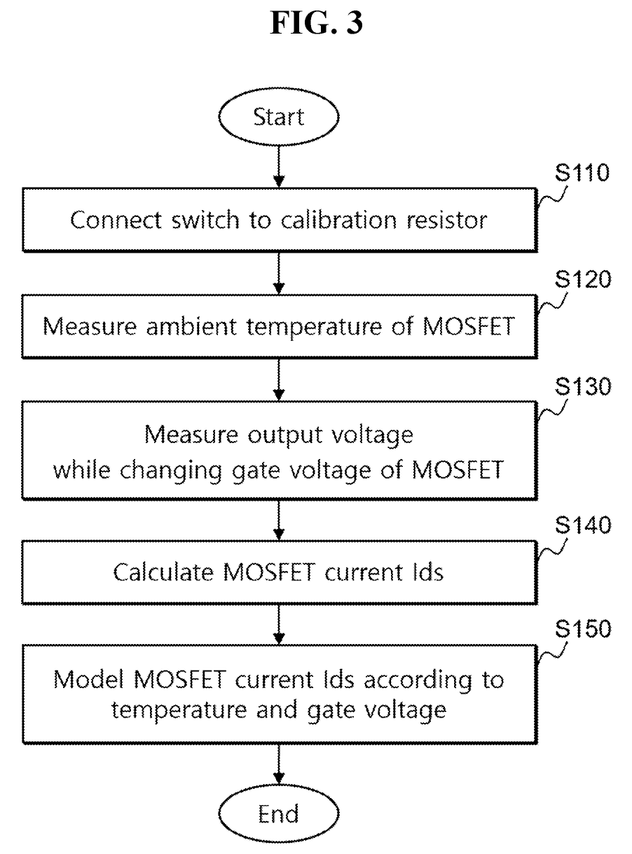

The MOSFET current modeling procedure step S100 is described in detail with reference to FIG. 3. FIG. 3 is a flowchart illustrating the process of modeling a MOSFET current that flows into skin with respect to an ambient temperature “t” and the gate voltage Vgs applied to the MOSFET 130 according to an embodiment of the present disclosure. An error according to the ambient temperature of the MOSFET 130 is calculated through the process of FIG. 3.

For the modeling, the controller 110 connects the switch 141 to the calibration resistor 142 (step S110) and measures the ambient temperature “t” through the temperature measuring unit 120 (S120). The controller 110 measures the output voltage Vout for the entire operating range of the MOSFET 130 while changing the gate voltage Vgs of the MOSFET 130 (step S130). For example, when the range of the gate voltage for a normal operation of the MOSFET 130 is between a minimum 2 V and a maximum 3 V, the controller 110 applies the gate voltage Vgs while raising the gate voltage by a predetermined unit voltage (e.g., 0.05 V) from 2 V to 3 V, and measures the output voltage Vout at that time. Furthermore, the controller 110 calculates the MOSFET current Ids based on the measured output voltage Vout and calibration resistance Rc of the calibration resistor 142 (step S140). That is, it is possible to obtain the MOSFET current Ids of the applied gate voltage by calculating the MOSFET current Ids based on the output voltage Vout and the calibration resistance Rc for the applied gate voltage. The measured and calculated value is stored in memory.

A nonlinear relation of the MOSFET current Ids according to the gate voltage of the MOSFET 130 is modeled by applying polynomial-based modeling and a parameter estimation method to data stored in the memory (step S150). If a set of parameters that is finally derived is used, a current model into which a manufacturing deviation of the MOSFET has been incorporated can be obtained. It is possible to estimate an accurate MOSFET current Ids by incorporating a current temperature “t” and the gate voltage Vgs applied to the MOSFET by using the current model obtained through the procedure of FIG. 3.

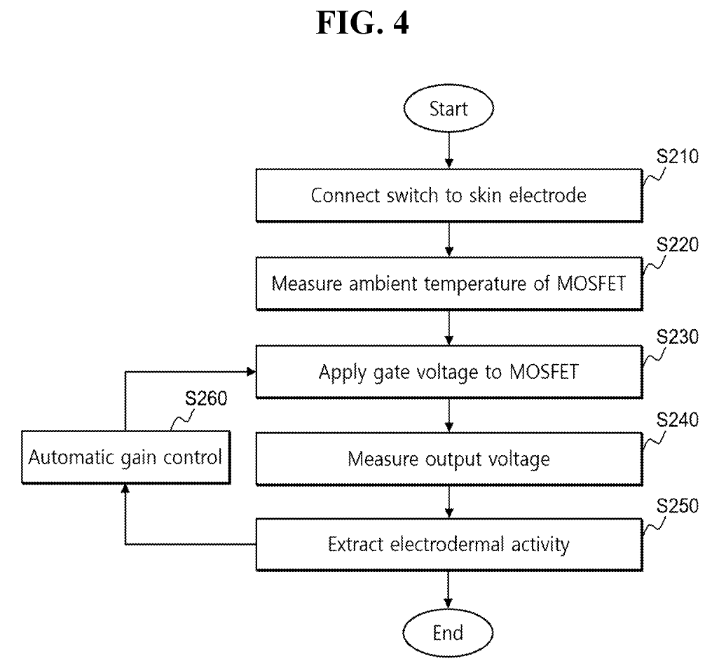

Next, the process (step S200) of measuring the electrodermal activity is described in detail with reference to FIG. 4. FIG. 4 illustrates a process of calculating the MOSFET current Ids that flows into the skin and extracting electrodermal activity based on the output voltage Vout by using model parameters obtained through the process of FIG. 3 with respect to the ambient temperature “t” that is measured in real time and the gate voltage Vgs applied to the MOSFET.

The controller 110 selects the skin electrodes 143 by connecting the switch 141 to the skin electrodes 143 (step S210), and measures the ambient temperature “t” of the MOSFET 130 in real time by using the temperature measuring unit 120 (step S220). The controller 110 applies the gate voltage Vgs as a voltage within the operating range of the MOSFET 130 (step S230), and measures the output voltage Vout that is output (step S240). The controller 110 calculates the MOSFET current Ids based on the measured value and model parameters, and calculates electrodermal activity that is measured in the skin through the skin electrodes 143 based on the measured voltage Vout and the calculated MOSFET current Ids (step S250). The controller 110 adjusts the gate voltage Vgs that is applied to the gate of the MOSFET based on the measured output voltage Vout (step S260), and measures electrodermal activity according to the gate voltage that is adjusted by performing step S230 to step S250. As described above, the controller 110 measures the electrodermal activity through automatic gain control so that an optimal gain is obtained while repeating the operation from step S230 to step S260.

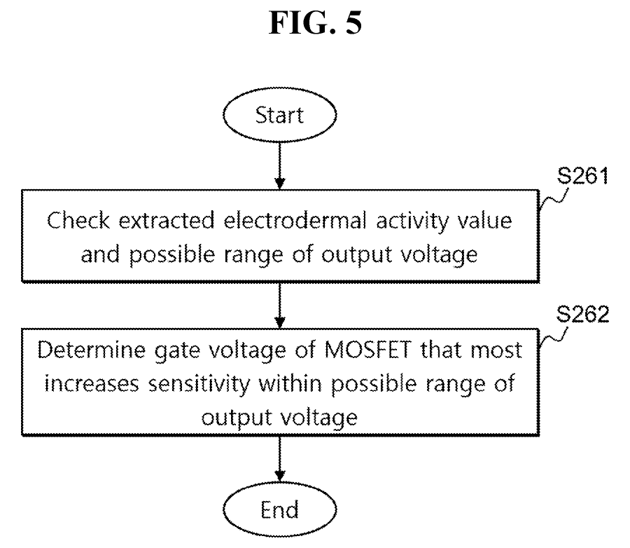

FIG. 5 is a flowchart illustrating a process of adjusting the gate voltage applied to the gate of the MOSFET 130 in order to adjust an optimal sensitivity and range based on an extracted electrodermal activity value. A problem that occurs in the process of measuring the electrodermal activity is solved and optimal signal sensitivity is obtained through the process of FIG. 5. The MOSFET current Ids of the MOSFET that flows into a circuit is adjusted based on the gate voltage Vgs that is applied to the gate of the MOSFET 130, and thus the gain of the non-inverting amplifier is adjusted. If the gain is increased, there is an advantage in that the sensitivity of electrodermal activity is improved, but an error may occur because a phenomenon in which the sensitivity of electrodermal activity is saturated in the output voltage Vout occurs. In order to prevent the occurrence of the error, the output voltage Vout is checked (step S261). The voltage gate Vgs that is applied to the gate of the MOSFET is adjusted so that the gain is adjusted in an optimal range of the output voltage Vout, for example, in a possible range of 50% to 150% of the output voltage Vout while increasing the sensitivity of electrodermal activity (step S262). According to an embodiment, the electrodermal activity may also be checked in step S261 and incorporated into a determination of an optimal range of the output voltage Vout.

Furthermore, the method according to an embodiment of the present disclosure may be implemented in the form of a program instruction which may be executed through various computer means, and may be recorded on a computer-readable medium.

The computer-readable medium may include a program instruction, a data file, and a data structure alone or in combination. A program instruction recorded on the computer-readable medium may be specially designed and constructed for an embodiment of the present disclosure or may be known and available to those skilled in the computer software field. The computer-readable medium may include a hardware device configured to store and execute the program instruction. For example, the computer-readable medium may include magnetic media such as a hard disk, a floppy disk, and a magnetic tape, optical media such as CD-ROM and a DVD, magneto-optical media such as a floptical disk, ROM, RAM, and flash memory. The program instruction may include not only a machine code produced by a compiler, but a high-level language code capable of being executed by a computer through an interpreter.

The embodiments of the present disclosure have been described in detail, but the scope of rights of the present disclosure is not limited thereto. A variety of modifications and changes made by those skilled in the art using the basic concept of the present disclosure defined in the appended claims are also included in the scope of rights of the present disclosure.

### DESCRIPTION OF REFERENCE NUMERALS

110: controller, 120: temperature measuring unit, 130: MOSFET, 140: amplification circuit unit, 141: switch, 142: calibration resistor, 143: skin electrodes, 144: amplifier.

What is claimed is:

1. An apparatus for measuring electrodermal activity, comprising:
a temperature measuring unit comprising a temperature sensor for measuring an ambient temperature;
an amplification circuit unit configured to amplify a signal;
a MOSFET configured to input a current to the amplification circuit unit; and
a controller configured to apply a gate voltage to a gate of the MOSFET based on the ambient temperature from the temperature measuring unit and an output voltage of the amplification circuit unit,
wherein the controller measures the output voltage of the amplification circuit unit, estimates a MOSFET current that flows from a drain of the MOSFET to a source of the MOSFET based on the applied gate voltage, and calculates electrodermal activity based on the measured output voltage and the estimated MOSFET current.

2. The apparatus of claim 1, wherein the amplification circuit unit comprises:
an amplifier;
a calibration resistor connected in parallel to the amplifier and for current modeling;
skin electrodes attached to skin and configured to measure electrodermal activity; and
a switch configured to select one of the calibration resistor and the skin electrodes,
wherein the controller connects the switch to the calibration resistor, generates a current model, and measures the electrodermal activity by using the current model by connecting the switch to the skin electrodes.

3. The apparatus of claim 2, wherein the controller generates the current model by performing:
connecting the switch to the calibration resistor,
measuring the ambient temperature through the temperature measuring unit,
applying the gate voltage to a gate of the MOSFET while changing the gate voltage and measuring the output voltage,
calculating the MOSFET current based on the measured output voltage and resistance of the calibration resistor, and
modeling a nonlinear relation of the MOSFET current to the gate voltage of the MOSFET, based on the measured ambient temperature, the applied gate voltage, the output voltage measured with respect to the applied gate voltage, and the calculated MOSFET current.

4. The apparatus of claim 3, wherein the controller applies the gate voltage to the gate of the MOSFET while raising the gate voltage by a predetermined unit voltage with respect to an entire operating range of the MOSFET and measures the output voltage at that time.

5. The apparatus of claim 4, wherein the controller uses polynomial-based modeling and a parameter estimation method when modeling the nonlinear relation of the MOSFET current to the gate voltage of the MOSFET.

6. The apparatus of claim 2, wherein the controller measures the electrodermal activity by performing:
a step of selecting the skin electrodes by connecting the switch to the skin electrodes,
a step of measuring the ambient temperature by using the temperature measuring unit,
an output voltage measuring step of applying the gate voltage within an operating range of the MOSFET and measuring the output voltage,
an electrodermal activity calculation step of calculating the MOSFET current based on the measured value and the current model, and calculating the electrodermal activity based on the measured output voltage value and the calculated MOSFET current,
a gate voltage adjustment step of adjusting the gate voltage based on the measured output voltage, and
a step of measuring the electrodermal activity while repeating the output voltage measuring step, the electrodermal activity calculation step, and the gate voltage adjustment step.

7. The apparatus of claim 6, wherein the controller checks the calculated electrodermal activity and adjusts the gate voltage so that a gain is adjusted to a predetermined range of a current signal while increasing a sensitivity of the electrodermal activity, in the gate voltage adjustment step.

8. The apparatus of claim 7, wherein the predetermined range of the current signal is a range of 50% to 150% of the current signal.

9. The apparatus of claim 4, wherein the controller measures the electrodermal activity by performing:
a step of selecting the skin electrodes by connecting the switch to the skin electrodes,
a step of measuring the ambient temperature by using the temperature measuring unit,
an output voltage measuring step of applying the gate voltage within an operating range of the MOSFET and measuring the output voltage,
an electrodermal activity calculation step of calculating the MOSFET current based on the measured value and the current model, and calculating the electrodermal activity based on the measured output voltage and the calculated MOSFET current,
a gate voltage adjustment step of adjusting the gate voltage based on the measured output voltage, and
a step of measuring the electrodermal activity while repeating the output voltage measuring step, the electrodermal activity calculation step, and the gate voltage adjustment step.

10. The apparatus of claim 9, wherein the controller checks the calculated electrodermal activity and adjusts the gate voltage so that a gain is adjusted to a predetermined range of a current signal while increasing a sensitivity of the electrodermal activity, in the gate voltage adjustment step.

11. A method of measuring electrodermal activity in an apparatus for measuring electrodermal activity, the apparatus comprising a temperature measuring unit comprising a temperature sensor for measuring an ambient temperature, an amplification circuit unit configured to amplify a signal, a MOSFET configured to input a current to the amplification circuit unit, and a controller configured to apply a gate voltage to a gate of the MOSFET based on the ambient temperature from the temperature measuring unit and an output voltage of the amplification circuit unit, and the amplification circuit unit comprising an amplifier, a calibration resistor for current modeling, skin electrodes attached to skin and configured to measure electrodermal activity, and a switch configured to select one of the calibration resistor and the skin electrodes, wherein the method comprises:
a current model generation step of connecting, by the controller, the switch to the calibration resistor and generating a current model, and
an electrodermal activity measuring step of connecting, by the controller, the switch to the skin electrodes and measuring electrodermal activity by using the current model.

12. The method of claim 11, wherein in the electrodermal activity measuring step, the controller connects the switch to the skin electrodes, applies the gate voltage to a gate of the MOSFET, measures the output voltage of the amplification circuit unit, estimates MOSFET current that flows from a drain of the MOSFET to a source of the MOSFET by using the current model based on the applied gate voltage, and calculates the electrodermal activity based on the measured output voltage and the estimated MOSFET current.

13. The method of claim 12, wherein the current model generation step comprises:
a step of connecting the switch to the calibration resistor,
a step of measuring the ambient temperature through the temperature measuring unit,
an output voltage measuring step of applying the gate voltage to the gate of the MOSFET while changing the gate voltage and measuring the output voltage,
a step of calculating the MOSFET current based on the measured output voltage and resistance of the calibration resistor, and
a modeling step of modeling a nonlinear relation of the MOSFET current to the gate voltage of the MOSFET based on the measured ambient temperature, the applied gate voltage, the output voltage measured with respect to the applied gate voltage, and the calculated MOSFET current.

14. The method of claim 13, wherein in the output voltage measuring step, the controller applies the gate voltage to the gate of the MOSFET while raising the gate voltage by a predetermined unit voltage with respect to an entire operating range of the MOSFET and measures an output voltage at that time.

15. The method of claim 14, wherein in the modeling step, a nonlinear relation of the MOSFET current to the gate voltage of the MOSFET is modeled by using polynomial-based modeling and a parameter estimation method.

16. The method of claim 12, wherein the electrodermal activity measuring step comprises:
a step of selecting the skin electrodes by connecting the switch to the skin electrodes,
a step of measuring the ambient temperature by using the temperature measuring unit,
an output voltage measuring step of applying the gate voltage within an operating range of the MOSFET and measuring the output voltage,
an electrodermal activity calculation step of calculating the MOSFET current by using the measured value and the current model and calculating the electrodermal activity based on the measured output voltage and the calculated MOSFET current,
a gate voltage adjustment step of adjusting the gate voltage based on the measured output voltage, and
a step of measuring the electrodermal activity while repeating the output voltage measuring step, the electrodermal activity calculation step, and the gate voltage adjustment step.

17. The method of claim 16, wherein in the gate voltage adjustment step, the controller checks the calculated electrodermal activity and adjusts the gate voltage so that a gate is adjusted to a predetermined range of a current signal while increasing a sensitivity of the electrodermal activity.

18. The method of claim 17, wherein the predetermined range of the current signal is a range of 50% to 150% of the current signal.

19. The method of claim 14, wherein the electrodermal activity measuring step comprises:
a step of selecting the skin electrodes by connecting the switch to the skin electrodes,
a step of measuring the ambient temperature by using the temperature measuring unit,
an output voltage measuring step of applying the gate voltage within an operating range of the MOSFET and measuring the output voltage,
an electrodermal activity calculation step of calculating the MOSFET current by using the measured value and the current model and calculating the electrodermal activity based on the measured output voltage and the calculated MOSFET current,
a gate voltage adjustment step of adjusting the gate voltage based on the measured output voltage value, and
a step of measuring the electrodermal activity while repeating the output voltage measuring step, the electrodermal activity calculation step, and the gate voltage adjustment step.

20. The method of claim 19, wherein in the gate voltage adjustment step, the controller checks the calculated electrodermal activity and adjusts a gain so that the gain is adjusted to a predetermined range of the current signal while increasing a sensitivity of the electrodermal activity.
